(19) United States
(12) Patent Application Publication
(10) Pub. No.: US 2012/0165552 A1
(43) Pub. Date: Jun. 28, 2012

(54) 7,10,13-CYCLOHEXADECATRIEN-16-OLIDE, AND FLAVOR OR FRAGRANCE COMPOSITION, FRAGRANCE OR COSMETIC PRODUCT, FOOD PRODUCT OR BEVERAGE OR TOILETRY PRODUCT COMPRISING THE SAME

(75) Inventors: Akinori Ikegami, Hiratsuka-shi (JP); Arata Yoneshige, Hiratsuka-shi (JP); Kenji Saiki, Hiratsuka-shi (JP); Teruhiko Inaba, Hiratsuka-shi (JP); Toru Shimizu, Hiratsuka-shi (JP); Yukihiro Kawakami, Hiratsuka-shi (JP)

(73) Assignee: TAKASAGO INTERNATIONAL CORPORATION, Tokyo (JP)

(21) Appl. No.: 13/394,080

(22) PCT Filed: Sep. 2, 2010
(86) PCT No.: PCT/JP2010/065451

§ 371 Date: Mar. 2, 2012

(30) Foreign Application Priority Data
Sep. 2, 2009 (JP) 2009-202641

(51) Int. Cl. C07D 313/00

(52) U.S. Cl. 549/266

(57) ABSTRACT

Provided is a novel compound which is excellent in odor quality, has a natural feeling and a fruity feeling and has a musk aroma. The present invention relates to a compound represented by the following formula (1):

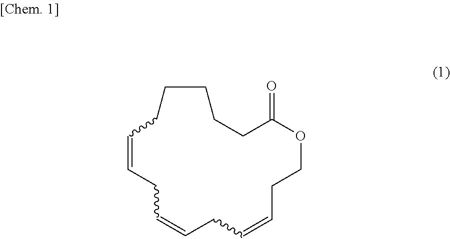

wherein wavy lines represent at least one of an E-configuration and Z-configuration of C═C double bond.

### TECHNICAL FIELD

The present invention relates to a novel macrocyclic lactone compound and a flavor or fragrance composition comprising said compound. In detail, it relates to the aforementioned compound having a musk aroma and a flavor or fragrance composition comprising said compound. It further relates to a fragrance or cosmetic product, food product or beverage or toiletry product which comprises said compound or said flavor or fragrance composition.

### BACKGROUND ART

Because of the difficulty in obtaining a natural musk flavor or fragrance from the viewpoint of animal protection and because of the recent uprush of nature directivity and serious consideration on environment, attention has been paid on a plant-derived compound having a musk aroma which has no problems regarding the safety, accumulation and degradation of the compound.

As the plant-derived musk flavor or fragrance so far known, exaltolide found from angelica root oil, ambrettolide which is present in ambrette seed oil and oxacycloheptadecan-2-one (cyclohexadecan-16-olide) found from cloudberry oil are known (see Non-patent Literature 1). In addition, as macrocyclic unsaturated lactones, 2E,7Z-cyclohexadecadien-16-olide, 2E,10E,12E-cycloheptadecatrien-17-olide and the like can be mentioned (see Non-patent Literature 2 and Non-patent Literature 3).

### CITATION LIST

### Non Patent Literature

NPL 1: Journal of Chromatography A, 787 (1997), p 276-282
NPL 2: Synthesis (1989), p 419-423
NPL 3: Asian Journal of Chemistry (2005), 17, p 859-870

### SUMMARY OF INVENTION

### Technical Problem

However, kinds of the plant-derived musk flavor or fragrance are still limited.

Accordingly, an object of the invention is to provide a novel compound which satisfies such demands, is excellent in odor quality and has a natural feeling and a fruity feeling and also has a musk aroma. Another object of the invention is to provide a flavor or fragrance composition which comprises the compound having the above-mentioned properties. Still another object is to provide a fragrance or cosmetic product, food product or beverage, and toiletry product which comprise said compound or said flavor or fragrance composition.

### Solution to Problem

With the aim of achieving the above-mentioned objects, the present inventors have conducted intensive studies and found as a result that a specific macrocyclic lactone compound in passion fruit has the above-mentioned properties and have accomplished the invention.

That is, the invention encompasses the following embodiments.

[1] A compound represented by the following formula (1), wherein wavy lines represent at least one of an E-configuration and Z-configuration of C═C double bond.

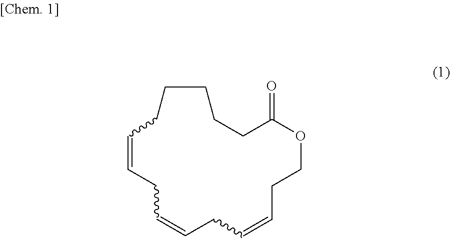

[2] The compound according to [1], which is 7Z,10Z,13Z-cyclohexadecatrien-16-olide.
[3] The compound according to [1] or [2], which has a musk aroma.
[4] A flavor or fragrance composition, which comprises the compound according to any one of [1] to [3].
[5] A product comprising the compound according to any one of [1] to [3], wherein the product is selected from the group consisting of a fragrance or cosmetic product, a food product or beverage and a toiletry product.
[6] A product comprising the flavor or fragrance composition according to [4], wherein the product is selected from the group consisting of a fragrance or cosmetic product, a food product or beverage and a toiletry product.

### Advantageous Effects of Invention

The compound of the invention, namely 7,10,13-cyclohexadecatrien-16-olide, has a musk aroma which is rich in a natural feeling and fruity feeling and, moreover, is excellent in diffusing property. Thus, the compound of the invention can be effectively used in various products such as a fragrance or cosmetic product, a food product or beverage and toiletry product, alone as such or in the form of a flavor or fragrance composition and, furthermore, can provide various products with the aforementioned excellent characteristics.

### DESCRIPTION OF EMBODIMENTS

The following describes embodiments of the invention in detail.

The compound of the invention, namely 7,10,13-cyclohexadecatrien-16-olide, is a compound represented by the following formula (1).

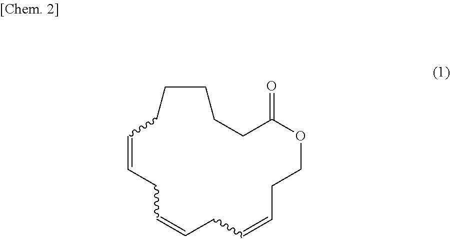

(In the formula, wavy lines represent at least one of an E-configuration and Z-configuration of C═C double bond.)

In the compound of the invention, all of the three C═C double bonds may be E-configuration, all of the three C═C double bonds may be Z-configuration, one of the three C═C double bonds may be E-configuration and two of the three C═C double bonds may be Z-configuration, or one of the three C═C double bonds may be Z-configuration and two of the three C═C double bonds may be E-configuration. In addition, the compound of the invention may be a mixture of two or more of the compounds with the above configurations.

As the compound of the invention, a Z-form compound represented by the following formula (2) (7Z,10Z,13Z-cyclohexadecatrien-16-olide) can be illustratively mentioned.

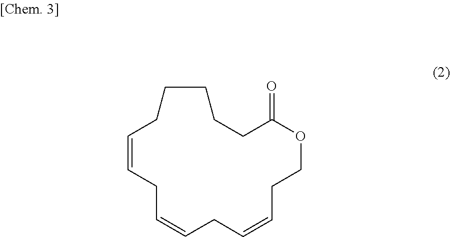

The compound of the invention is most suitably obtained from raw fruits of the passion fruit, but in addition to this, it can also be obtained from its leaves, stems, roots and processed products thereof (fruit juice, dry fruit, extract, solvent extract and the like). In addition, the raw fruits of the passion fruit can also be obtained from both of its yellow species and purple species.

The method for obtaining the compound of the invention from raw fruits of the passion fruit is described in the following. In this connection, the operations for obtaining the compound of the invention are not particularly limited, and any method can be used.

(1) Firstly, extraction is carried out by adding a solvent to raw fruits of the passion fruit. The raw fruits of the passion fruit can be subjected to the extraction by optionally removing seeds or pulping them. As the solvent for extraction, for example, there may be mentioned alcohols such as methanol, ethanol and propanol; halogenated hydrocarbons such as methylene chloride and chloroform; hydrocarbons such as pentane, hexane, benzene and toluene; ethers such as diethyl ether and tetrahydrofuran (THF); esters such as methyl acetate and ethyl acetate; acetonitrile, N,N-dimethylformamide (DMF), acetone, dimethyl sulfoxide (DMSO), water and the like, though not limited thereto. A mixed solvent prepared by mixing these solvents may be used. Among them, diethyl ether, ethyl acetate, methylene chloride and chloroform can be preferably mentioned. In addition, physical techniques such as agitation and maceration and chemical techniques such as pH adjustment may be employed at the time of the extraction.

The solvent to be used in the extraction is preferably from 0.01 to 1,000 times by weight, more preferably from 0.5 to 2 times by weight, based on the weight of raw fruits of the passion fruit. The extraction time is preferably from 0.01 hour to 1,000 hours, more preferably from 18 hours to 48 hours, and the extraction temperature is preferably from −116 to 140° C., more preferably from 5 to 30° C.

(2) After the extraction, the extract liquid is distilled at a liquid temperature of from 30 to 200° C., preferably at a liquid temperature of from 30 to 120° C., and under from normal pressure to 1 Pa, preferably from normal pressure to 10 Pa, thereby taking out fractions having musk aroma. When taking out the fractions having musk aroma, a sensory evaluation is carried out by three evaluators, and the fraction recognized as significant by two or more evaluators can be used (the same shall apply hereinafter).

(3) Next, the fraction obtained in the aforementioned (2) is fractionated using a silica gel column chromatography and an elution solvent, and a fraction having musk aroma is taken out. A commercial item can be used as the silica gel column chromatography, and for example, Silica gel 60, 70 to 230 meshes, (manufactured by Nacalai Tesque) can be mentioned. As the elution solvent, for example, there may be mentioned alcohols such as methanol, ethanol and propanol; halogenated hydrocarbons such as methylene chloride and chloroform; hydrocarbons such as pentane, hexane, benzene and toluene; ethers such as diethyl ether and tetrahydrofuran (THF); esters such as methyl acetate and ethyl acetate; acetonitrile, N,N-dimethylformamide (DMF), acetone, dimethyl sulfoxide (DMSO) and the like, though not limited thereto. In addition, these solvents may be used alone or a mixed solvent prepared by mixing them may be used. Among these solvents, a mixture of ethyl acetate and hexane can be preferably mentioned.

(4) Next, the fraction obtained in the aforementioned (3) is fractionated using a reverse phase thin-layer chromatography and an elution solvent, and a fraction having musk aroma is taken out. A commercial item can be used as the reverse phase thin layer chromatography, and for example, RP-18F₂₅₄ s, 1 mm (manufactured by Merck & Co., Inc.) can be mentioned. As the elution solvent, for example, there may be mentioned alcohol-based solvent such as methanol, ethanol and propanol; water, acetonitrile, acetone, tetrahydrofuran (THF) and the like, though not limited thereto. In addition, these solvents may be used alone or a mixed solvent prepared by mixing them may be used. Among these solvents, a mixture of water and methanol can be preferably mentioned.

(5) Next, the fraction obtained in the aforementioned (4) is fractionated using a high performance liquid chromatography, and a fraction having musk aroma is taken out. As the column of the high performance liquid chromatography, ODS can be mentioned and preferably, COSMOSIL (registered trademark) 5C18-AR-II (manufactured by Nacalai Tesque) can be mentioned. As the elution solvent, for example, there may be mentioned alcohol-based solvent such as methanol, ethanol and propanol; water, acetonitrile, acetone, tetrahydrofuran (THF) and the like, though not limited thereto. In addition, these solvents may be used alone or a mixed solvent prepared by mixing them may be used. Preferred among these solvents is a mixture of water and methanol.

In this manner, the compound of the invention, 7,10,13-cyclohexadecatrien-16-olide, can be obtained from fresh fruits of the passion fruit. Structure of the thus obtained compound can be identified by ¹H-NMR and ¹³C-NMR.

The compound 7,10,13-cyclohexadecatrien-16-olide of the invention has a musk aroma.

In addition, the compound of the invention can be directly blended as such in a fragrance or cosmetic product, a food product or beverage, a toiletry product and the like as a flavor or fragrance compound.

As the fragrance or cosmetic product, for example, there may be mentioned fragrance products such as perfume, eau de parfum, eau de toilette and eau de cologne; skin care products such as milky lotion, skin lotion, beauty lotion, pack and cream; styling preparations such as hair spray, hair wax, pomade, hair cream and set lotion; cosmetics such as foundation, face powder, rouge, lip balm and eye shadow; and detergent for clothing use, bleacher, air freshener, antiperspirant, body spray, and the like. The amount of the compound of the invention contained in the fragrance or cosmetic product can be set to a level of preferably from 0.0001 to 3% by weight, more preferably from 0.01 to 0.5% by weight, based on the total weight of fragrance or cosmetic product.

As the food product or beverage with which the compound of the invention can be blended, for example, there may be mentioned carbonated drinks, soft drinks, coffee drinks, fruits and fruits flavored drinks, dairy drinks, vegetable drinks; tea drinks such as green tea, roasted tea, Oolong tea and black tea; drinks such as alcohol; ices such as ice candies, ice creams and sherbets; confectionary such as candy, chocolate, chewing gum, jelly and snack food, and the like. The amount of the compound of the invention contained in the food product or beverage can be set to a level of preferably from 1×10⁻¹° to 1×10⁻²% by weight, more preferably from 1×10⁻⁷ to 1×10⁻³% by weight, based on the total weight of food product or beverage.

As the toiletry product with which the compound of the invention can be blended, for example, there may be mentioned shampoo, conditioner, hair growing agent, cleansing cream, body soap, soap, bath preparation, dentifrice and the like. The amount of the compound of the invention contained in the toiletry product can be set to a level of preferably from 0.0001 to 3% by weight, more preferably from 0.01 to 0.5% by weight, based on the total weight of toiletry product.

The compound of the invention can be prepared to a flavor or fragrance composition together with other flavor or fragrance component(s). As the other flavor or fragrance component(s) which can be contained together with the compound of the invention, for example, there may be mentioned various synthetic aroma chemicals such as esters, alcohols, aldehydes, ketones and lactones, materials of natural origin such as essential oil, oleoresin and extract, and the like.

In addition, for example, the flavor or fragrance components described in “Synthetic Aroma chemicals—Chemistry and Commodity Knowledge (written in Japanese)” (edited by M. Indo, published on Mar. 6, 1996, by The Chemical Daily Co., Ltd.), “Perfume and Flavor Chemicals (Aroma Chemicals) 1, 2” (Steffen Arctender (1969)) and the like can be used. As typical examples among them, linalool, α-terpineol, ethyl hexanoate, hexyl hexanoate, 2-methyl-3-buten-2-ol, hexanol, damascenone, β-ionone and the like can be mentioned.

The amount of the compound of the invention contained in the flavor or fragrance composition may be optionally decided in response to the kind of the flavor or fragrance composition and object and therefore is not particularly limited, but it may be good to set it to a level of generally from 1×10⁻⁸ to 50% by weight, preferably from 5×10⁻⁶ to 5% by weight, based on the entire flavor or fragrance composition.

In addition to the above-mentioned flavor or fragrance components, the flavor or fragrance composition of the invention can also be blended with generally used retaining agent and solvent. As the retaining agent, for example, glycerin, glyceride, alkylene glycol, alkyl citrate, benzyl benzoate and the like can be mentioned. As the solvent, for example, alcohol such as ethanol, water, propylene glycol, glycerin, triacetin and the like can be mentioned.

The flavor or fragrance composition of the invention can be blended in a fragrance or cosmetic product, a food product or beverage or a toiletry product. As the fragrance or cosmetic product, food product or beverage or toiletry product in which the flavor or fragrance composition can be blended, those which were described in the above can for example be mentioned. The amount of the flavor or fragrance composition of the invention contained in the fragrance or cosmetic product is preferably from 0.001 to 50% by weight, its amount contained in the food product or beverage is preferably from 1×10⁻⁷ to 1% by weight and its amount contained in the toiletry product is preferably from 0.001 to 50% by weight.

### EXAMPLES

The following describes the invention further illustratively with reference to examples, but the invention is not limited thereto and may be changed within such a range that it does not deviate from the scope of the invention. In this connection, the instruments shown below were used in the following for the measurement of physical property values and the like.

NMR measuring apparatus: Instrument DRX 500 (manufactured by BRUKER BIOSPIN K.K.)

Gas chromatograph mass spectrometer: GCMS-QP 2010 (manufactured by Shimadzu Corporation)

Gas chromatograph-olfactometer: HP-6890 manufactured by Agilent technologies

Liquid chromatograph mass spectrometer: LCMS-IT-TOF (manufactured by Shimadzu Corporation)

High performance liquid chromatograph
Pump: LC-10AT (manufactured by Shimadzu Corporation)
Detector: SPD-M10A (manufactured by Shimadzu Corporation)
Separation column: COSMOSIL (registered trademark) 5C18-AR-II (manufactured by Nacalai Tesque)

### Example 1

### Isolation of 7Z,10Z,13Z-cyclohexadecatrien-16-olide from raw fruits of passion fruit

After removing seeds from 20 kg of raw fruits of passion fruit (purple species), extraction was carried out at room temperature for 24 hours by adding 10 kg of diethyl ether. Next, diethyl ether was evaporated by carrying out distillation under normal pressure at a liquid temperature of 35° C., and high vacuum distillation was further carried out in order to remove low boiling point compounds. The distillation was completed when the degree of vacuum was 120 Pa and liquid temperature was 97° C. and when the overhead temperature was lowered from about 70° C. to about 50° C.

Subsequently, this was divided into 5 fractions by a silica gel column chromatography (elution solvent; hexane:ethyl acetate=98:2 in volume ratio) and each fraction was analyzed by a gas chromatography-olfactometry, thereby obtaining a fraction having a musk aroma at a retention time of from 30 to 45 minutes.

This fraction was further fractionated by a reverse phase thin-layer chromatography fractionation (developing solvent volume ratio, methanol:water=20:1). After the development, this was divided into 3 fractions based on the separation patterns (UV absorption) of the thin-layer chromatography, and the odor components of each fraction were extracted with methanol. The extracted odor components were analyzed by a gas chromatography-olfactometry, methanol in the fraction having a musk aroma was evaporated and then the residue was purified by a high performance liquid chromatography (elution solvent; methanol:water=8:2 (volume ratio)) to obtain 7Z,10Z,13Z-cyclohexadecatrien-16-olide (purity 94%).

### Physical data of 7Z,10Z,13Z-cyclohexadecatrien-16-olide

¹H NMR (500 MHz, CDCl₃) δ:

5.55 to 5.35 (6H, m), 4.16 (2H, t, J=5.89), 2.85 (2H, t, J=6.70), 2.81 (2H, t, J=6.32), 2.41 to 2.38 (2H, m), 2.29 (2H, t, J=7.12), 2.08 to 2.04 (2H, m), 1.67 to 1.62 (2H, m), 1.37 to 1.32 (4H, m)

¹³C NMR (126 MHz, CDCl₃) δ:

173.9, 131.3, 129.7, 128.6, 128.0, 128.0, 125.9, 63.5, 34.4, 28.3, 27.8, 27.5, 26.7, 25.9, 25.7, 24.6; HRMS (ESI+) calcd C₁₆H₂₅O₂ (MH⁺), 249.1849. found, 249.1851

### Gas Chromatography Analysis:

Column: BC-WAX (50 m×0.25 mm×0.15 μm; manufactured by GL Science), measuring temperature: from 70° C. to 218° C. (temperature rising at 4.0° C./min), retention time=40.69 min

### Gas Chromatography-Olfactometry Analysis:

BC-WAX (50 m×0.25 mm×0.15 μm; manufactured by GL Science), measuring temperature: from 70° C. to 218° C. (temperature rising at 4.0° C./min)

### Example 2

### Isolation of 7Z,10Z,13Z-cyclohexadecatrien-16-olide from raw fruits of passion fruit

7Z,10Z,13Z-cyclohexadecatrien-16-olide was able to be obtained in the same manner as in Example 1, except that the raw fruits of the passion fruit were changed to those of the yellow species.

### Test Example 1

### Sensory Evaluation

By carrying out odor evaluation by 7 professional panelists on the 7Z,10Z,13Z-cyclohexadecatrien-16-olide isolated in Example 1, exaltolide (cyclopentadecanolide (manufactured by Firmenich)) and ambrettolide (manufactured by Symrise), a 3-grade (A: strong, B: usual, C: weak) relative evaluation on respective odor patterns (musk, fruity, natural and diffusing property) was carried out. The results are shown in Table 1.

TABLE 1

| | | | Compound | | |
|---|---|---|---|---|---|
| | | | of | | |
| Sample name | | | Example 1 | Exaltolide | Ambrettolide |
| Odor | Musk | A (strong) | 4 | 3 | 0 |
| patterns | | B (usual) | 3 | 3 | 5 |
| | | C (weak) | 0 | 1 | 2 |
| | Fruity | A (strong) | 5 | 0 | 0 |
| | | B (usual) | 2 | 3 | 5 |
| | | C (weak) | 0 | 4 | 2 |
| | Natural | A (strong) | 6 | 0 | 0 |
| | | B (usual) | 1 | 3 | 5 |
| | | C (weak) | 0 | 4 | 2 |
| | Diffusing | A (strong) | 6 | 1 | 1 |
| | property | B (usual) | 1 | 5 | 3 |
| | | C (weak) | 0 | 1 | 3 |

Each numeral represents the number of panelists

Based on the results of Table 1, 7Z,10Z,13Z-cyclohexadecatrien-16-olide had a fruity musk aroma, was rich in diffusing property and showed strong natural feeling, in comparison with those of exaltolide and ambrettolide.

### Example 3

### Preparation of Flavor or Fragrance Composition

A flavor or fragrance composition was prepared using the 7Z,10Z,13Z-cyclohexadecatrien-16-olide of the invention and in accordance with the prescription shown in the following Table 2.

TABLE 2

| | Parts by weight |
|---|---|
| Ethyl acetate | 11 |
| Cis-3-hexenol | 3 |
| Hexanol | 5 |
| Benzaldehyde | 2 |
| Ethyl hexanoate | 22 |
| Cis-3-hexenyl acetate | 2 |
| Linalool | 70 |
| Linalool oxide | 4 |
| Hexyl butyrate | 25 |
| Hexyl hexanoate | 15 |
| α-Terpineol | 5 |
| Citral | 15 |
| Cis-3-hexenyl butyrate | 4 |
| Cis-3-hexenyl hexanoate | 5 |
| β-Ionone | 6 |
| Ethyl butyrate | 10 |
| 8-Mercaptomenthone | 0.01 |
| 7Z,10Z,13Z-Cyclohexadecatrien-16-olide | 0.01 |
| 95% Ethanol | 795.98 |
| Total amount | 1000 |

### Example 4

### Preparation of Flavor or Fragrance Composition

A flavor or fragrance composition was prepared using the 7Z,10Z,13Z-cyclohexadecatrien-16-olide of the invention and in accordance with the prescription shown in the following Table 3.

TABLE 3

| | Parts by weight |
|---|---|
| Allyl cyclohexylpropionate | 3 |
| Cassis Base | 1 |
| Tricyclodecenyl propionate | 10 |
| δ-Damascenone | 1 |
| Decalactone | 10 |
| Dimethylbenzylcarbinyl propionate | 10 |
| Methyl dihydrojasmonate | 150 |
| Hexyl salicylate | 100 |
| 4-t-Butyl benzyl alcohol | 200 |
| Linalool | 80 |
| γ-Methyl ionone | 30 |
| MUSK T (registered trademark) | 110 |
| HINDINOL (registered trademark) | 5 |
| o-tert-Butylcyclohexyl acetate | 50 |
| 7Z,10Z,13Z-Cyclohexadecatrien-16-olide | 40 |
| Total amount | 800 |

MUSK T (registered trademark): ethylene brassylate, manufactured by Takasago International Corporation
HINDINOL (registered trademark): 2-methyl-4-[(1R)-2,2,3-trimethyl-3-cyclopenten-1-yl]-(2E)-buten-1-ol, manufactured by Takasago International Corporation

### Example 5

### Preparation of Carbonated Drink

A carbonated drink (Brix 9.3, acidity 0.13% (in terms of citric acid), pH 3.4, gas volume 3.0) was prepared using the flavor or fragrance composition prepared in Example 3 and in accordance with the prescription shown in the following Table 4. The thus obtained carbonated drink was suitably possessed of a musk aroma and a natural feeling.

TABLE 4

| | Parts by weight |
|---|---|
| High-fructose corn syrup | 93.9 |
| Granulated sugar | 20.0 |
| Citric anhydride | 1.3 |
| Flavor or fragrance composition prepared in Example 3 | 1.0 |
| Sodium citrate | 0.1 |
| Water | 200 |
| Carbonated water | 783.7 |
| Total amount | 1100 |

### Example 6

### Preparation of Liquid Bath Preparation

A liquid bath preparation was prepared using the flavor or fragrance composition prepared in Example 4 and in accordance with the prescription shown in the following Table 5. The thus obtained liquid bath preparation was suitably possessed of a musk aroma.

TABLE 5

| | Weight % |
|---|---|
| 1,3-Butylene glycol | 2.00 |
| Polyoxyethylene (40) hydrogenated castor oil | 0.75 |
| Polyoxyethylene sorbitan monooleate (20 EO) | 0.75 |
| 95% Ethanol | 40 |
| 3-(1-Menthoxy)propane-1,2-diol | 3.00 |
| Flavor or fragrance composition prepared in Example 4 | 1.00 |
| Pigment | Proper amount |
| Purified water | Balance |
| Total amount | 100 |

### Example 7

### Preparation of Shampoo

A shampoo was prepared using the flavor or fragrance composition prepared in Example 4 and in accordance with the prescription shown in the following Table 6. The thus obtained shampoo was suitably possessed of a musk aroma.

TABLE 6

| | Weight % |
|---|---|
| Purified water | 41.83 |
| POIZ C-60H*¹ | 0.60 |
| EMAL 20C*² | 40.00 |
| BEAULIGHT A-5000*³ | 5.00 |
| SWANOL AM-101*⁴ | 5.00 |
| AMIZOL CDE*⁵ | 4.00 |
| Glycerin | 0.10 |
| Ethylene glycol distearate | 1.50 |
| Citric acid | 0.22 |
| Potassium chloride | 0.30 |
| Methyl paraben | 0.20 |
| Propyl paraben | 0.10 |
| Ethyl paraben | 0.10 |
| Tetrasodium edetate | 0.05 |
| Flavor or fragrance composition prepared in Example 4 | 1.00 |
| Total amount | 100 |

*¹o-[2-hydroxy-3-(trimethylammonio)propyl]hydroxy-ethylcellulose chloride
*²sodium polyoxyethylene lauryl ether sodium sulfate (3 E.O) (25%)
*³disodium polyoxyethylene lauroyl ethanol amide sulfosuccinate (5 E.O)
*⁴2-alkyl-N-carboxymethyl-N-hydroxyethylimidazolinium betaine (40%)
*⁵coconut oil fatty acid diethanolamide

### Example 8

### Preparation of Deodorant Powder Spray (Antiperspirant)

A deodorant powder spray was prepared using the flavor or fragrance composition prepared in Example 4 and in accordance with the prescription shown in the following Table 7. The thus obtained deodorant powder spray was suitably possessed of a musk aroma.

TABLE 7

| | Weight % |
|---|---|
| Chlorohydroxy aluminum | 1.00 |
| Silicic anhydride | 1.00 |
| Isopropyl myristate | 2.00 |
| Octamethylcyclotetrasiloxane | 0.50 |
| Sorbitan sesquioleate | 0.10 |
| Triclosan | 0.02 |
| 3-(1-Menthoxy)propane-1,2-diol | 1.50 |
| Flavor or fragrance composition prepared in Example 4 | 0.20 |
| 95% Ethanol | 3.33 |
| LPG | 90.35 |
| Total amount | 100.00 |

While the invention has been described in detail and with reference to specific embodiments thereof, it will be apparent to one skilled in the art that various changes and modifications can be made therein without departing from the spirit and scope thereof. This application is based on Japanese Patent Application No. 2009-202641 filed on Sep. 2, 2009, and their contents are incorporated herein by reference.

### INDUSTRIAL APPLICABILITY

The compound of the invention, namely 7,10,13-cyclohexadecatrien-16-olide, has a musk aroma which is rich in a natural feeling and fruity feeling and, moreover, is excellent in diffusing property. Thus, the compound of the invention can be effectively used in various products such as a fragrance or cosmetic product, a food product or beverage and toiletry product, alone as such or in the form of a flavor or fragrance composition and, furthermore, can provide various products with the aforementioned excellent characteristics.

1. A compound represented by the following formula (1): 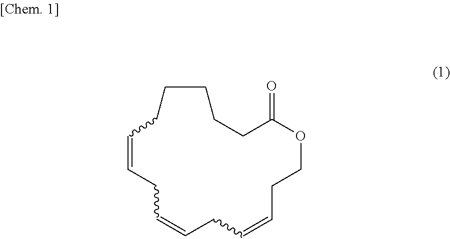
wherein wavy lines represent at least one of an E-configuration and Z-configuration of C═C double bond.

2. The compound according to claim 1, which is 7Z,10Z,13Z-cyclohexadecatrien-16-olide.

3. The compound according to claim 1, which has a musk aroma.

4. A flavor or fragrance composition, which comprises the compound according to claim 1.

5. A product comprising the compound according to claim 1, wherein the product is selected from the group consisting of a fragrance or cosmetic product, a food product or beverage and a toiletry product.

6. A product comprising the flavor or fragrance composition according to claim 4, wherein the product is selected from the group consisting of a fragrance or cosmetic product, a food product or beverage and a toiletry product.

7. The compound according to claim 2, which has a musk aroma.

8. A flavor or fragrance composition, which comprises the compound according to claim 2.

9. A flavor or fragrance composition, which comprises the compound according to claim 3.

10. A flavor or fragrance composition, which comprises the compound according to claim 7.

11. A product comprising the compound according to claim 2, wherein the product is selected from the group consisting of a fragrance or cosmetic product, a food product or beverage and a toiletry product.

12. A product comprising the compound according to claim 3, wherein the product is selected from the group consisting of a fragrance or cosmetic product, a food product or beverage and a toiletry product.

13. A product comprising the compound according to claim 7, wherein the product is selected from the group consisting of a fragrance or cosmetic product, a food product or beverage and a toiletry product.

14. A product comprising the flavor or fragrance composition according to claim 8, wherein the product is selected from the group consisting of a fragrance or cosmetic product, a food product or beverage and a toiletry product.

15. A product comprising the flavor or fragrance composition according to claim 9, wherein the product is selected from the group consisting of a fragrance or cosmetic product, a food product or beverage and a toiletry product.

16. A product comprising the flavor or fragrance composition according to claim 10, wherein the product is selected from the group consisting of a fragrance or cosmetic product, a food product or beverage and a toiletry product.
